(19) United States
(12) Patent Application Publication
(10) Pub. No.: US 2026/0284127 A1
(43) Pub. Date: Sep. 24, 2026

(54) COMPOSITIONS OF BIOACTIVES IN CORDYCEPS AND METHODS OF DELIVERY FOR THE TREATMENT OF ACUTE AND LONG COVID

(71) Applicant: SINOVEDA CANADA INC., Edmonton (CA)

(72) Inventors: Yun Kau TAM, Edmonton (CA); Chih-yuan TSENG, Edmonton (CA)

(21) Appl. No.: 19/475,537

(22) PCT Filed: Apr. 17, 2024
(86) PCT No.: PCT/CA2024/050501

§ 371 Date: Oct. 15, 2025

Related U.S. Application Data
(60) Provisional application No. 63/496,538, filed on Apr. 17, 2023.
(60) Provisional application No. 63/496,611, filed on Apr. 17, 2023.

(51) Int. Cl. A61K 36/068; A61K 31/7076; A61P 31/14

(52) U.S. Cl. CPC A61K 36/068; A61K 31/7076; A61P 31/14

(57) ABSTRACT

This invention discloses a composition of active ingredients found in the genus of cordyceps, in defined ratios, the combination of ingredients, when administered orally in an appropriate dosage form, are effective in the treatment of COVID and Long COVID.

### BACKGROUND OF THE INVENTION

Since WHO announced COVID-19 as a pandemic three years ago (Mar. 11, 2020), 680 million cases were reported, and 1% (6.8 million) of those infected died. As of January 2023, WHO maintain that COVID-19 is still a global emergency although the world is entering a transition point.

Long COVID is the crisis after COVID. It has long term impacts on health and economy. In the United States alone, 70% of Americans contracted COVID. Twenty-four percent of these subjects experienced symptoms for 3 months or more. More than 16 million working age individuals have long COVID. Annual lost wages surpass $170 billion USD, the figure could be as high as $230 billion USD.

There is no consensus of the etiology of Long COVID, let alone effective treatments. There are several hypotheses for the cause of the condition. These include but are not limited to residual viruses or remnants of the virus left in the body, the overactive immune system has not returned to normal, micro-clots in the blood and tissue and organ damage caused by the virus.

There are more than 100 symptoms associated with Long COVID; the most common ones are fatigue, tiredness or loss of energy (72.1%), cough (39.3%), shortness of breath (38.5%), brain fog (32.9%) and general weakness (30.9%) (https://health-infobase.canada.ca/covid-19/post-covid-condition/).

The COVID infection effects on the body, especially the lung, brain, and the gastrointestinal tract, immune, and the circulatory system have been well described.

Symptoms may vary due to the progression of the disease, particularly in relation to the viral load, the imbalance immune system, and the damage to the body.

There is a void and an unmet need for a drug or treatment for the dreadful conditions associated with the sickness. Cordyceps spp. are known for antiviral, anti-inflammatory and immunomodulating properties (Jedrejko, Lazur et al. 2021, Rabie 2022). Recently, an extract of Cordyceps militaris has been tested in patients suffering from COVID (Dubhashi, Sinha et al. 2023). Unfortunately, the efficacy was marginal.

It has been suggested that bioactive compounds in Genus Cordyceps could have an effect in treating COVID (Kaymakci and Guler 2020, Verma 2020, Zivan, Ruiz et al. 2023). However, in the quantities identified, and the common doses consumed (3 to 6 g of fungus per day), none of these compounds, when tested individually, showed therapeutic implications. These include adenosine and cordycepin (Verma 2020, Zivan, Ruiz et al. 2023). Clinical efficacy of cordyceps polysaccharides has not been established although its anti-inflammatory and immunomodulating activities are well documented (Miao, Yu et al. 2022).

For example, while cordycepin is more effective than remdesivir (Rabie 2022), the required daily dosage of cordycepin is higher than 450 mg when administered alone. This would mean that 450 grams of Cordyceps will be required, assuming the cordycepin level in the fungus is 1%. It is not practical in terms of dietary or pharmaceutical application.

### SUMMARY OF THE INVENTION

In this disclosure, a combination treatment derived from Cordyceps is described. Without restriction to a theory, it is believed that the individual components have synergistic interactions. As a result, an acceptable dose size has been developed, and preliminary human testing shows that at least one embodiment described herein may be effective in treating COVID-19 and/or long COVID.

Aspects of the present invention were developed at least in part with data mining techniques to describe the disease network of COVID, and Long COVID.

In one aspect, disclosed is a pharmaceutical composition comprising a combination of cordycepin, adenosine and polysaccharide extracted from Cordyceps fungi. Preferably, the cordycepin and adenosine are also extracted from Cordyceps fungi.

In another aspect, disclosed are oral dosage forms comprising a composition described herein, which when administered to a subject in an effective amount, provides effective treatment of COVID and/or long COVID.

In another aspect, disclosed is a method of treating COVID and/or long COVID, comprising administering a combination of cordycepin, adenosine and polysaccharide extracted from Cordyceps fungi to a patient, in a therapeutically effective amount.

In another aspect, disclosed is a use of a composition to treat COVID and/or long COVID, the composition comprising a combination of cordycepin, adenosine and polysaccharide extracted from Cordyceps fungi.

Embodiments of the invention may comprise any aspect apparent from this description and any combination of features or elements described herein.

### BRIEF DESCRIPTION OF THE FIGURES

FIG. 1A. Pathways that regulate the production of cytokine and hypoxia. FIG. 1B. Pathways that regulate the innate immune system, coagulation, and platelet activity. In both figures, the 3 components are represented by grey ovals labeled by their generic names. Potential targets are highlighted in light grey color. Large font size is used for the target genes associated with pathways that are likely regulated by these compounds. A line connecting two boxes with an arrow at the end denotes activation; a line with a T at the end denotes inhibition; a solid line without end symbol denotes binding/association; a dash line with an arrow denotes indirect interaction, and a dotted line represents uncertain interactions. Besides, the width of a line between a compound (oval) and a target (rectangle box) represents efficacy, 1 (thickest), the most potent, to 30 (thinnest), the least potent, the unit of concentration is μM. Finally, we use 10 grey solid and dash lines to highlight the downstream signaling pathways of targets that are likely to be regulated by the compounds.

FIG. 2. Simulated 24-hour concentration profile in human plasma, lung tissue, and muscle after orally administering 1 mg of cordycepin.

FIG. 3. A minimum effective dose of cordycepin required to inhibit 50% of SARS-CoV-2 virus in each organ.

FIG. 4. A representative HPLC/DAD chromatogram of a sample of SCI-2213. The identity of the compounds is confirmed using the overlay of this chromatogram with their respective standards. Red: Standard; Blue: SCI-2213 Extract.

FIG. 5. An HPLC/DAD trace of the hydrolyzed SCI-2213 polysaccharides. 1-phenyl-3-methyl-5-pyrazolone (PMP) was used as the derivatizing agent. As reported in the literature, glucose is the most abundant monosaccharide in the polysaccharides.

FIG. 6: Poly Acrylamide Gel Electrophoresis Separation of Polysaccharides (PS-PAGE) from Cordyceps militaris.

FIG. 7: Comparison of Caco-2 cell permeability of pure cordycepin vs cordycepin in SCI-2213. The quantity of cordycepin is expressed as percentage of original concentration added to the apical side (% Detected).

FIG. 8. Demographics of participants involved in the SCI-2213 study.

FIG. 9. Distribution of volunteers who participated in the study in terms of days after positive COVID diagnosis.

FIG. 10. Treatment response (n=40) to 11 common COVID and long COVID symptoms.

FIG. 11. The average number of doses to produce the significant response to the 11 COVID and long COVID symptoms.

### DETAILED DESCRIPTION

### Terminology

The transitional terms “comprising”, “consisting essentially of”, and “consisting” are intended to connote their generally in accepted meanings in the patent vernacular; that is, (i) “comprising”, which is synonymous with “including”, “containing”, or “characterized by”, is inclusive or open-ended and does not exclude additional, unrecited elements or method steps; (ii) “consisting of” excludes any element, step, or ingredient not specified in the claim; and (iii) “consisting essentially of” limits the scope of a claim or embodiment to the specified materials or steps “and those that do not materially affect the basic and novel characteristic(s)” of the claimed invention or the embodiment. More specifically, the basic and novel characteristics relates to the ability of the method or use to provide at least one of the benefits described herein, including but not limited to the ability to improve the survivability of the human population relative to the survivability of the comparative human population described elsewhere herein. Embodiments described in terms of the phrase “comprising” (or its equivalents), also provide, as embodiments, those which are independently described in terms of “consisting of” and “consisting essentially of”.

When a value is expressed as an approximation by use of the descriptor “about”, it will be understood that the particular value forms another embodiment. If not otherwise specified, the term “about” signifies a variance of ±10% of the associated value, but additional embodiments include those where the variance may be ±5%, ±15%, ±20%, ±25%, or ±50%, in particular the term “about” signifies a variance of ±5% or ±10% of the associated value, more in particular ±5%.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used herein, the singular forms “a,” “an,” and “the” include the plural.

As used herein, “patient” is intended to mean any animal, in particular, mammals. Thus, the methods or uses are applicable to human and nonhuman animals, although preferably with humans. The terms “patient” and “subject” and “human” may be used interchangeably.

The terms “treat” and “treatment” refer to the treatment of a patient afflicted with a pathological condition and refers to an effect that alleviates the condition through any mechanism of action, but also to an effect that results in the inhibition of the progress of the condition, and includes at least one of a reduction in the rate of progress, a halt in the rate of progress, amelioration of the condition, relief of symptoms, prophylaxis against infection or symptoms, and cure of the condition.

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

The term “dosage” refers to the information of the amount of the therapeutic to be taken by the subject and the frequency of the number of times the therapeutic is to be taken by the subject. The term “dose” refers to the amount or quantity of the therapeutic to be taken each time.

The term “COVID” as used herein refers to a severe acute respiratory syndrome (SARS) caused by a virus known as SARS-Coronavirus 2 (SARS-CoV2). The term “Long COVID” refers to symptoms which persist, for example, for more than 12 weeks after the initial infection with the virus. Long COVID may also be known as Post-COVID-19 syndrome.

The terms “co-administration” or the like, as used herein, encompass administration of the selected therapeutic agents to a single patient, and are intended to include treatment regimens in which the agents are administered by the same or different route of administration or at the same or different time.

The term “pharmaceutical combination” as used herein, means a product that results from the mixing or combining of more than one active ingredient and includes both fixed and non-fixed combinations of the active ingredients. The term “fixed combination” means that the active ingredients, e.g., polysaccharides, adenosine and cordycepin, are both administered to a patient simultaneously in the form of a single unit or single dosage form. The term “non-fixed combination” means that the active ingredients, e.g., polysaccharides, adenosine and cordycepin are administered to a patient as separate units or separate dosage forms, either simultaneously, concurrently or sequentially with no specific intervening time limits, wherein such administration provides safe and effective levels of the active ingredients in the body of the human. The latter also applies to cocktail therapy, e.g., the administration of three or more active ingredients.

As used herein, “cordyceps polysaccharide” or “polysaccharides” means long-chain polymeric carbohydrates composes of monosaccharide units linked by glycosidic bonds which may be extracted from Cordyceps fungi, including exopolysaccharides, intracellular polysaccharides, and acidic polysaccharides.

Cordycepin or 3′-deoxyadenosine, is a derivative of the nucleoside adenosine, differing from the latter by the replacement of the hydroxy group in the 3′ position with a hydrogen. It may be extracted from the Cordyceps fungi, but may also be produced synthetically.

Adenosine is a nucleoside, and is known as a pharmaceutical ingredient, particularly for the treatment of some cardiac arrhymthmias. Adenosine may be extracted from many different sources, including Cordyceps fungi, but may also be produced synthetically.

In the history of Traditional Chinese Medicine (TCM), cordyceps, usually in the form of Cordyceps sinensis, has been used as an adaptogen and medicine. More than 600 species of Cordyceps have been described in the genus. The most common species used in TCM are C. sinensis and C. militaris.

Cordyceps sinensis are typically found in high altitude. Over harvesting has made the cost of C. sinensis skyrocket. Natural C. militaris is even rarer, and its active ingredient profile is better than that of C. sinensis. Since the natural supply of Cordyceps is not sustainable, these two species are being cultivated, and the chemical profiles of cultivated cordyceps have been shown to be similar to that found in nature.

This specification discloses a composition comprising cordycepin, adenosine and a Cordyceps fungal extract comprising polysaccharides, preferably in the appropriate ratios and dosages described herein. In preferred embodiments, the cordycepin and adenosine components are also extracted from a Cordyceps fungus. Embodiments of this composition were administered to a group of subjects diagnosed with COVID and long COVID. Post infection period ranged from one to over 800 days.

Pharmaceutical compositions provided herein comprise an effective amount of the synergistic combination. The phrases “pharmaceutical or pharmacologically acceptable” refers to molecular entities and compositions that do not produce an adverse, allergic or other untoward reaction when administered to an animal, such as, for example, a human, as appropriate, or where an adverse, allergic or other untoward reaction may result but is tolerable in view of a therapeutic benefit to be gained. The preparation of a pharmaceutical composition comprising the synergistic combination will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by a regulatory body.

In some embodiments, the composition may be provided in a dosage form, preferably an oral dosage form, which provides a daily dose of polysaccharides of between about 200 to about 800 mg, a daily dose of cordycepin between about 15 to about 50 mg, and a daily dose of adenosine is between about 4 to about 50 mg, for a human having a body mass of 70 kg. Suitable daily doses may be calculated per kg of body mass for any particular individual. For example a 200-400 mg capsule, may comprise 1.2 to 15.0 mg of adenosine, 5.0 mg to 15.0 mg of cordycepin and 50 mg to 200 mg of polysaccharides. A daily dose may be taken once, or divided and taken twice or three times a day, or more, as is known in the art.

Suitable oral dosage forms are well-known in the art, and include capsules, tablets, solutions, suspensions, powder, and nano-encapsulated dosage forms.

In some preferred embodiments, the ratio of polysaccharides to cordycepin may be between about 3:1 to about 40:1, and the ratio of cordycepin to adenosine may be between about 1:5 to about 10:1.

The pharmaceutical composition may comprise a pharmaceutically acceptable carrier, which includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions disclosed herein is contemplated.

Exemplary compositions were developed using the systems biology approach developed by Tam, Tseng et al. (2022). The following examples describe the sequence of the developmental process and the test of a formula in human. In summary, the chemical profiles of Cordyceps sinensis/militaris were compiled and potential active compounds are identified.

Compound-target interactions in the documented COVID disease pathways were identified. The number of bioactive compounds with potential therapeutic values was filtered using criteria for drug-like properties and the abundance of the active compounds in the fungus. As a result, three bioactive compounds, adenosine, cordycepin and polysaccharides were identified.

In this specification, anti-viral, anti-inflammatory and immunomodulating activities of cordycepin and its mechanism of actions are described.

In this specification, the anti-viral activity of adenosine is described.

In In this specification, the anti-viral effects of cordyceps polysaccharides through immune-modulation and anti-inflammatory activities are described.

In this specification, the drug-like properties of cordycepin, adenosine and polysaccharides are described in humans using in silico estimation or human pharmacokinetic data.

In this specification, effective oral doses of the combination therapy are determined for the treatment of COVID and Long COVID.

The anti-COVID and anti-long COVID effects of SCI-2213 were tested in 40 human subjects. SCI-2213 demonstrated significant effects in alleviating 11 common symptoms of COVID and long COVID.

The following examples are intended to illustrate certain embodiments or aspects of the invention disclosed herein, and not to be limiting of the claimed invention.

### Example 1

The objective of this example is to use the methodology developed by Tam, Tseng et al. (2022) to first map out the disease network of COVID, followed by evaluating compounds in Cordyceps sinensis/militaris that could have effects on targets and pathways which are responsible for the disease. Subsequently, the compound-target network is mapped out, hence, potential mechanisms of action of these compounds are obtained.

It is assumed that long COVID symptoms are similar to COVID except the severity of the disease and symptoms are different in that more damage to the body by the virus are present in patients with long COVID; however, the virus load in long COVID patients is lower.

### Disease Network of COVID and Long COVID

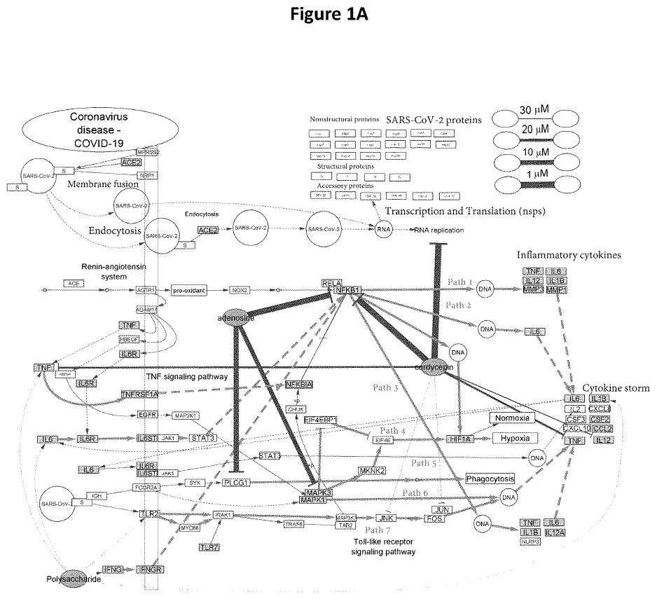

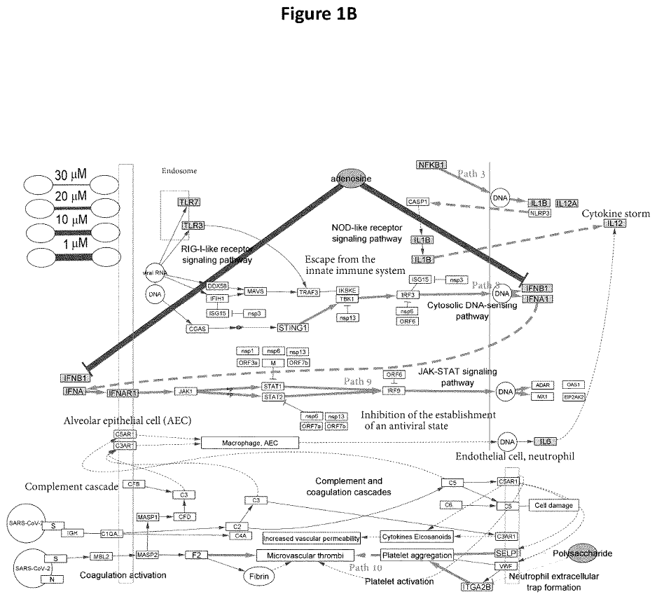

The disease network of COVID is summarized in KEGG (KEGG ID, HSA:05171) and their pathways are described in FIGS. 1A and 1B. The network consists of 14 signaling pathways through which the SARS-CoV-2 virus proteins trigger inflammatory cytokines, cytokine storm, inflammation, coagulation, evasion of innate immune systems and inhibition of the establishment of an antiviral state. In addition to the primary pathways described in KEGG, targets which are either directly or indirectly involved in regulating the primary pathway of the COVID network are also taken into consideration.

The targets involved in the disease process are outlined below:
Immunomodulation, targets include Interleukin 12 (IL-12), and Interferon γ (IFNγ).
Inflammation, targets include IL-1α, IL-1β, IL-6, IL-10, Tumor necrosis factor a (TNFα), Transforming growth factor β (TGFβ1), IFNγ.
Hypoxia tolerance, targets include Nuclear factor erythroid 2-related factor 2 (Nrf2), Hypoxia-inducible factor 1 (HIF1), Nuclear factor kappa B subunit 1 (NFκB), and Angiotensin-converting enzyme 2 (ACE2).
Fibrosis includes pulmonary fibrosis, targets include two families, Matrix metalloproteinases (MMPs)/Tissue inhibitors of metalloproteinases (TIMPs), IFNγ, CSF2, ELANE, CCL2, CXCR4, and IL-1α.

Targets associated with the severity of COVID-19 infection are (Karlowitz, Stanifer et al. 2022, Lu, Shirvani et al. 2022, Namkoong, Edahiro et al. 2022, Redin, Thorball et al. 2022):
Dedicator of cytokinesis 2 (DOCK2), the key target (Namkoong, Edahiro et al. 2022).
IFNα, IFN1, IFNαR, IFNαR2, IFN1R
Non-receptor tyrosine-protein kinase (TYK2)
Toll-like receptors TLR3 and TLR7
Neutrophils/Lymphocytes, targets include Folate receptor γ (FOLR3) and Regulator of G-protein signaling 1 (RGS1).
   Identification of Active Compounds in Cordyceps sinensis/militaris

In this example, we focus on Cordyceps sinensis/militaris, which have been reported to have multiple functions in immunomodulation, anti-viral, anti-inflammatory, and antioxidant activities. And therefore, these two fungi may contain ingredients which are active against COVID-19. The chemical profiles of cordyceps are collected from TM-MC (https://informatics.kiom.re.kr/compound/search.do). A total of 91 compounds have been identified in cordyceps (Table 1).

TABLE 1
The list of 91 unique compounds methyladenosine

| | | | | | |
|---|---|---|---|---|---|
| 2′-O- | 2′-deoxyadenosine | 2′-deoxyguannosine | 2′-deoxyuridine | 2-chloroadenosine | 22-dihydroergosteryl- |
| methyladenosine | | | | | beta-D-glucopyranoside |
| 3′-deoxyadenosine | 5-(hydroxymethyl)- | CSP1 | CSP1-2 | D-mannitol | N6-(2- |
| | 1-(2-oxopiperidin- | | | | hydroxyethyl) adenosine |
| | 3-y1)-1H-pyrrole-2- | | | | |
| | carbaldehyde | | | | |
| adenine | adenosine | adenosine-5′- | arachidonic acid | ascorbic acid | beta-sitosterol |
| | | monophosphate | | | |
| campesterol | cerevisterol | cholesterol | cholesteryl | cinnamaldehyde | cordycepin |
| | | | palmitate | | |
| cordyglucan | cordylagenin | cordyrrole A | cordyrrole B | cordysinan | cyclo (Gly-Pro) |
| cyclo-Ala-Leu | cyclo-Ala-Leu-rha | cytidine | cytosine | deoxyadenosine-5′- | deoxyribonuclease |
| | | | | monophosphate | |
| dihydrouracil | docosanoic acid | ergosterol | ergosteryl-3- | exopolysaccaride | galactomannan |
| | | | beta-D- | | |
| | | | glucopyranoside | | |
| glucose | guanine | guanosine | guanosine 5′- | hesperidin | heteroglycan |
| | | | monophosphate | | |
| hypoxanthine | inosine | isoergotamine | lauric acid | lignoceric acid | limonin |
| linoleic acid | linoleyl acetate | mannitol | myriocin | myristic acid | nicotinamide |
| oleic acid | palmitic acid | palmitoleic acid | pentadecanoic | phenylalanine | phenylalanine-O-glu |
| | | | acid | | |
| peroxyergosterol | rugulosin | rutin | skyrin | stearic acid | styrone |
| thiamine | thymidine | thymine | trehalose | uracil | uridine |
| uridine-5′- | uralene | vernine | xanthine | xanthosine | beta-thymidine |
| monophosphate | | | | | |
| gamma-Aminobutyric | Ergothioneine | glycolipids | lutein | zeaxanthin | lovastatin |
| acid | | | | | |
| polysaccharides | | | | | |

Using the PK/PD criteria for the hit analysis, 17 chemicals were identified from cordyceps that might interact with targets involved in the COVID-19 disease network. They include cordycepin, adenosine, 2-chloroadenosine, 2′-O-methyladenosine, adenosine-5′-monophosphate (AMP), guanosine 5′-monophosphate (GMP), campesterol, cholesterol, lovastatin, and 8 fatty acids: pentadecanoic acid, docosanoic acid, lauri acid, myristic acid, lignoceric acid, stearic acid, palmitic acid, and linoleic acid. Prior to constructing the compound-target interaction network, biological assays result of these 17 compounds were reviewed. The 8 fatty acids reported in PubChem are activities of chemical derivatives of these compounds, hence, these 8 fatty acids were excluded.

Yang et al. reported that Cordyceps militaris contains small amounts of AMP and GMP, in amounts from about 100 to 700 μg/g (Yana. Li et al. 2010). Although studies suggested that AMP and GMP have promising effects in pharmacology, they are not included in the network construction due to the insignificant quantities present. These compounds are also not detected in the extract disclosed in this invention.

Similarly, 2-chloroadenosine, 2′-O-methyladenosine, campesterol, cholesterol, and lovastatin are also not detected in the cordyceps extract disclosed in this invention. Therefore, these compounds are also excluded from the compound-target network construction.

Although our automatic data mining did not include polysaccharides, many studies have suggested these groups of compounds possess potential activities against Covid-19 (Kuo, Chen et al. 2007, Ohta Y. 2007, Lee, Kwon et al. 2015, Liu, Feng et al. 2016, Mao, Song et al. 2022). Note that these studies have used polysaccharides including exopolysaccharides, intracellular polysaccharides, and acidic polysaccharides. Therefore, we included all these polysaccharides in our investigations. However, we will simply label them as polysaccharide in addition to the remaining 2 chemicals to construct the interaction network. The roles of these 3 chemicals are summarized in Table 2.

TABLE 2
Summary of compound-target interaction network analysis in COVID-19.

| | | Signaling | | |
|---|---|---|---|---|
| Functions | Target | pathways | Compounds | Remarks |
| Regulation | NFκB1 | Renin- | Cordycepin, Adenosine; | Inhibition |
| of | | angiotensin | | |
| cytokine | | system | | |
| storm | MAPK3 | Fc γ R-mediated | Adenosine, | Inhibition |
| | | phagocytosis | | |
| | TNF | Inflammatory | Cordycepin | Inhibition |
| | | cytokine | Polysaccharides | Increased |
| | | | | expression |
| | IL1β | Inflammatory | Polysaccharides | Increased |
| | | cytokine | | expression |
| | IL6 | Inflammatory | Polysaccharides | Increased |
| | | cytokine | | expression |
| | IL12 | Inflammatory | Cordycepin | Inhibition |
| | | cytokine | | |
| | IFNγ | HIF-1 | Polysaccharides | Increased |
| | | | | expression |
| | JNK, JUN, | Toll-like | Cordycepin | Inhibition |
| | FOS | receptor | Lovastatin | Activation |
| | TLR2 | Toll-like | Polysaccharides | Increased |
| | | receptor | | expression |
| Regulation | IFNβ1/ | Cytosolic DNA- | Adenosine | Inhibition |
| of innate | IFNα1 | sensing | | |
| immune | PLCγ 1 | Fc γ R-mediated | Adenosine | Inhibition |
| system | | phagocytosis | | |
| Decreasing | NκB1 | Renin- | Cordycepin, Adenosine; | Inhibition |
| the risk | | angiotensin | | |
| of | | system | | |
| fibrosis | MAPK3 | Fc γ R-mediated | Adenosine, | Inhibition |
| | | phagocytosis | | |
| Decreasing | CD62p | Complement | Polysaccharides | Inhibition |
| the risk | (selectin) | cascade | | |
| of | | | | |
| thrombosis | | | | |
| Increasing | MAPK1 | HIF-1 | Adenosine, | Require |
| hypoxia | NFκB1 | HIF-1 | Cordycepin, Adenosine | further |
| tolerance | | | | studies to |
| | | | | unveil true |
| | | | | activities. |

Cordyceps-COVID19 interaction network. In addition to Table 2, FIGS. 1(A) and (B) illustrate the interaction network of the 3 compounds and their potential targets in the COVID-19 disease network. FIG. 1(A) presents the pathways that regulate the production of cytokine and hypoxia. FIG. 1(B) presents the pathways that regulate the innate immune system, coagulation, and platelet activity. In both figures, the 3 compounds are represented by grey ovals labeled by their generic names. Potential targets, addressed earlier, are highlighted by light grey color. Large font size is used for the target genes associated with pathways that are likely regulated by these compounds. A line connecting two boxes with an arrow at the end denotes activation, a line with a T at the end denotes inhibition, a solid line without end symbol denotes binding/association, a dash line with an arrow denotes indirect interaction, and a dotted line represents uncertain interactions. The width of a line between a compound (oval) and a target (rectangle box) represents activity values ranging from 1 (thickest) to 30 (thinnest) ρM. In other words, the thicker the line the stronger the activity of the compound. Finally, we use 10 grey solid and dash lines to highlight the downstream signaling pathways of targets that are likely regulated by the three compounds.

Potential mechanisms of action of cordyceps in treating COVID-19. Given this interaction network (FIGS. 1(A) and (B)), a comprehensive picture of how these three chemicals might regulate cellular functions which are involved in treating COVID-19. The following are the potential roles of these three chemicals in the hallmarks of COVID-19.
Regulation of inflammatory cytokines. Since SARS-CoV-2 virus utilizes ACE2 to enter cells, it has a direct effect on renin angiotensin system. One of the downstream targets is NFκB1, which leads to the expression of several inflammatory cytokines (Path 1 of FIG. 1(A)) and provides one route to trigger cytokine storm. Two chemicals, cordycepin and adenosine, likely inhibit NFκB1, and could reduce the expression of inflammatory cytokines.
Regulation of cytokine storms. There are multiple routes that can regulate cytokine storms; they include the renin angiotensin system, TNF signaling pathway, Fc γ R-mediated phagocytosis, Toll-like receptor signaling pathway, and Nod-like receptor signaling pathway. Since the renin angiotensin system has been discussed, subsequent disclosure will focus on the rest of the pathways. Note that it was found that polysaccharides may increase the expression of IL-1β.
   Regarding the TNF signaling pathway, it primarily promotes the expression of several inflammatory cytokine genes through NFκB1 to mediate the innate immune system (Path 1, 2, and 3 of FIG. 1(A)). Two chemicals, cordycepin and adenosine, would likely inhibit NFκB1 and reduce the expression of inflammatory cytokines. However, polysaccharides were found to increase TNF and IFNγ gene expression and promote NFκB1 expression while cordycepin inhibits both.
   Regarding Fc γ R-mediated phagocytosis, it promotes the expression of inflammatory cytokine genes through MAPK (Path 6 of FIG. 1(A)). Adenosine may inhibit MAPK which mediates cytokine storms.
   Regarding the Toll-like receptor signaling pathway (Path 7 of FIG. 1(A)), there are mixed effects from the three compounds. Cordycepin was found to inhibit JNK and JUN (AP-1 transcription factor) and suppresses cytokine storms. On the other hand, polysaccharides were found to increase expression of TLR-2, which may promote downstream cytokine genes expression.
   Regarding the Nod-like receptor signaling pathway, it promotes the innate immune system through an indirect activation by NFκB1, which can be downregulated through Toll-like receptor pathway (Path 3 of FIG. 1(A)). Again, the two chemicals, adenosine and cordycepin, that inhibit NFκB1 can also mediate the innate immune system through this route.
Regulation of phagocytosis. Fc γ R-mediated phagocytosis is one of the immune responses in humans to resolve viral infections. Our mining indicates that adenosine may suppress Fc γ R-mediated phagocytosis through inhibiting PLCγ1 (Path 5 of FIG. 1(A)).
Regulation of fibrosis. As disclosed earlier, several targets, including MMPs, CSF2, and CCL2, are associated with fibrosis (Kaymakci and Guler 2020). Our mining found that cordycepin and adenosine, may either directly or indirectly suppress the expression of these genes through inhibiting several upstream targets in Renin-angiotensin system, Fc γ R-mediated phagocytosis and Nod-like receptor signaling pathways (Path 1, 2, 3, and 6 of FIG. 1(A)). Therefore, they may be able to regulate fibrosis.
Regulation of hypoxia. Several targets including Nrf2, HIF1, NFκB1, and ACE2 are associated with hypoxia tolerance as shown in Path 4 of FIG. 1(A). As suggested by (Singh, Tulsawani et al. 2013) in their in vitro studies, the cells treated with Cordyceps sinensis result in an increase of Nrf2 and HIF1 level and a decrease of NFkB1, which significantly improve hypoxia tolerance. Based on the data mining disclosed herein, NFκB1 is the only target found to be directly inhibited by cordycepin and adenosine. However, it was suggested that polysaccharides may increase NFκB1 expression through activating IFNγ and IL6. Yet the inhibition of NFκB1 may reduce the expression of HIF1. In addition, HIF1 may be regulated by adenosine through the inhibition of MAPK1 as shown in Path 4 of FIG. 1(A). However, since MAPK1 also inhibits the translation of HIF1 through inhibiting EIF4EBP1, the overall activities of these 2 chemicals in increasing the expression of HIF1 require further investigations. Despite the lack of clarity on the effects of the three active compounds on hypoxia, and the potential effects of polysaccharides on hypoxia prevention was reported by Dong, Hu et al. (2015).
Regulation of the innate immune system. It has been shown that several SARS-CoV-2 viral proteins including nsp3, nsp6 and ORF6 may help the virus escape from the innate immune system by suppressing several targets in the cytosolic DNA sensing pathway (Path 8 of FIG. 1(B)). Unfortunately, the present disclosure did not render any compounds having potential to prevent SARS-CoV-2 virus escape from the immune system.
Regulation of the anti-viral state establishment. The JAK-STAT signaling pathway plays an essential role in regulating inflammation in response to viral infection. Particularly, the interferons stimulated genes (ISGs), which involve a DNA binding component IRF9, (FIG. 1(B)). These ISGs act at different stages of the viral life cycle leading to an antiviral state that provides adequate immunity against viruses. It has been shown that the absence of IRF9 leads to an impaired antiviral state (Wang, Xu et al. 2017). As shown in FIG. 1(B), SARS-CoV-2 virus inhibits the establishment of an antiviral state by inhibiting STAT and IRF9. Our mining identified one chemical, adenosine, that could regulate the JAK-STAT signaling pathway (Path 9 of FIG. 1(B)). Unfortunately, adenosine was found to inhibit IFNβ, which could suppress the establishment of the antiviral state.
Regulation of thrombosis. One of the complications associated with COVID-19 is thrombophilia (Badulescu, Sirbu et al. 2022). As shown in the Complement cascade in the bottom of FIG. 1(B), SARS-CoV-2 virus is likely to promote overexpression of thrombin in the blood system and increases the risk of thrombosis. It was found that polysaccharides may inhibit platelet aggregation and decrease the risk of thrombosis through inhibiting CD62p and ITGA2B (Path 10 of FIG. 1(B)). The anti-platelet activity of Cordyceps polysaccharides was demonstrated by Mao, Song et al. (2022).

Cordyceps has been shown to have beneficial effects in treating COVID-19. We identified three compounds that could be responsible for these effects. These compounds' potential mechanisms of action were investigated using the compound-target interaction networks (FIGS. 1(A) and (B)). Our findings suggest that these compounds may regulate patients' intracellular signaling to activate the immune system against SARS-CoV-2 virus and/or restore multiple functions affected by viruses. The potential mechanisms of action of these compounds against COVID-19 are summarized by five key elements: 1. Regulation of cytokine storms, 2. Enhancement of the immune system, 3. Increase hypoxia tolerance, 4. Decrease the risk of thrombosis, and 5. Decrease the risk of fibrosis. As shown in Table 2, these five key elements are elaborated as follows:
Regulation of cytokine storms: There are several routes to regulate cytokine storms. Cordycepin and adenosine can suppress cytokine storms by inhibiting two targets NFκB1 and MAPK. By downregulating different pathways to suppress the expression of cytokines, these chemicals have shown potential in reducing inflammatory responses.
Enhancement of the immune system: Our findings suggest that polysaccharides are promising compounds to enhance the immune system through regulating TNF and Toll-like receptor pathways by activating TNF, IL6, TLR2, and IFNγ.
Increase hypoxia tolerance: Our findings indicate that cordycepin and adenosine may have dual effects in regulating the expression of HIF1, a key Biomarker in regulating hypoxia. Depending on the specific pathway involved, these two chemicals can either activate or inhibit hypoxia-related pathways. By understanding these mechanisms more, we can potentially increase hypoxia tolerance. Furthermore, the contribution of polysaccharides to increase hypoxia tolerance needs to be elucidated.
Decrease the risk of thrombosis: Polysaccharides may decrease the risk of thrombosis by inhibiting platelet activation through inhibiting integrin alpha II beta. Again, by understanding these mechanisms more, the potential risk associated with thrombosis can be decreased.
Decrease the risk of fibrosis: According to our findings, no compound was able to directly inhibit one of the fibrosis associated genes, MMP3. Other fibrosis-associated genes, such as CSF2 and CCL2, can only be regulated indirectly by inhibiting NFκB1 and MAPK by cordycepin and adenosine.

In summary, our studies suggest that cordycepin and adenosine play dual roles in regulating cytokine production and the immune system, which are crucial components in COVID-19 treatment. On one hand, cordycepin and adenosine can suppress cytokine storms, but on the other hand, they may also suppress the adaptive immune system. Polysaccharides, on the other hand, may activate TNFα, IL-6, TLR2, and IFNγ to counteract the effects of cordycepin and adenosine, achieving a balance in the immune system. Additionally, polysaccharides may also play a role in regulating platelet activity and reducing the risk of thrombosis. By combining cordycepin, adenosine, and cordyceps polysaccharides, preferably in effective proportions, and with effective dosages, it is believed without restriction to a theory, that the immune system may be regulated to combat COVID-19 and long COVID effectively.

### Example 2

The objective of this example is to estimate the minimum clinically effective dose of cordycepin in humans using in silico methods.

In the recent meta-analysis, remdesivir was shown to reduce mortality in patients hospitalized with COVID-19 who required no conventional oxygen support (Amstutz, Speich et al. 2023). According to both in vitro and in silico studies, cordycepin demonstrated a greater inhibitory effect on key SARS-CoV-2 protein targets-including the spike (S) protein, main protease (M(pro)) enzyme, and RNA-dependent RNA polymerase (RdRp) enzyme-compared to remdesivir (Rabie 2022). However, the pharmacokinetics of cordycepin in humans are not well elucidated. The objectives of this example are to estimate the pharmacodynamic (PD) and pharmacokinetic (PK) parameters of cordycepin computationally, with the aim to estimate a human dose, through PK/PD simulation, which can be utilized to efficiently manage COVID-19 and provide relief from long COVID.

Using Vero E6 cells with the first variant of SARS-CoV-2 strain from December 2020, Rabie (2022) reported an IC₅₀ value of 2 ρM or 0.502 μg/ml for cordycepin. This IC₅₀ value is adopted as the PD parameter for the current dosage estimation. The remaining goal is to estimate the minimum effective dose of cordycepin required to inhibit SARS-CoV-2.

PK simulation. We used PK-Sim program from Open systems Pharmacology© suite to conduct PBPK (physiologically based pharmacokinetic) simulations. At the time of this simulation, we identified only one clinical report using an herbal formula, HAD-B1, which contains cordycepin. One goal in the study was to determine the pharmacokinetics of cordycepin in HAD-B1 formula. Cordycepin concentration changes in 24 hours after one oral dose of the formula in a subject was collected and analyzed. Unfortunately, the data is not yet released except time to maximal concentration (1.5 hours) and half-life (8.62 hours). In addition to this, we also obtained experimental solubility in water from Sigma's product information sheet, which is 1 mg/ml and CaCo2 permeability data, which is 1.1*10⁻⁷ cm/s (Lee, Radhi et al. 2019). Therefore, we use these data to prepare the required input parameters to establish a PK model for humans. Note that tissue to plasma coefficients were estimated using the method of Poulin and Theil (2002). The default values from PK-Sim for the rest of physiological parameters for a 30-year-old male with body weight 73 kg were used. In the simulation, a single dose of 1 mg of cordycepin was orally administered. The 24-hr concentration profiles of cordycepin in plasma and lung tissue were generated and replotted using Originpro.

Method for estimating minimum effective dose. Since effective doses EC₅₀ (ng/ml) of a compound of interests can be interpreted as the concentration required to have 50% of chances to either activate or inhibit certain cellular functions, it suggests that the concentration level of the compound in plasma over T hours needs to be greater than or at least equal to EC₅₀*T to produce effects on target cells at site of actions. Namely, given a compound of interest with initial dose D₀ (mg) that will have a 50% chance to either activate or inhibit target proteins in human over T hours, we need to have

$${AUC}_{0 - T}^{{D}_{0}}$$

drug concentration in plasma,

$${AUC}_{0 - T}^{{D}_{0}} / T \geq E {C}_{50} . \qquad ( 1 )$$

Since we can approximate

$${AUC}_{0 - T}^{{D}_{0}}$$

by

$${AUC}_{0 - T}^{{D}_{0}} ≅ {D}_{0} {AUC}_{0 - T}^{1} , \qquad ( 2 )$$

where

$${AUC}_{0 - T}^{1}$$

is the drug concentration in plasma over T hours when 1 mg of drug is orally administrated, substituting Eq. (2) into (1) gives:

$${D}_{0} \geq E {C}_{50} / \overline{{AUC}^{1}} \qquad ( 3 )$$

where

$${\overline{AUC}}_{0 - T}^{1} = {AUC}_{0 - T}^{1} / ( 1 * T ) .$$

Therefore, the lower boundary in Eq. (3) can be defined as the minimum effective dose of this compound,

$${D}_{min} {=}^{E {C}_{50}} / {\overline{AUC}}_{0 - T}^{1} \qquad ( 4 )$$

Eq. (4) will be the working equation in our analysis shown in the Results section.

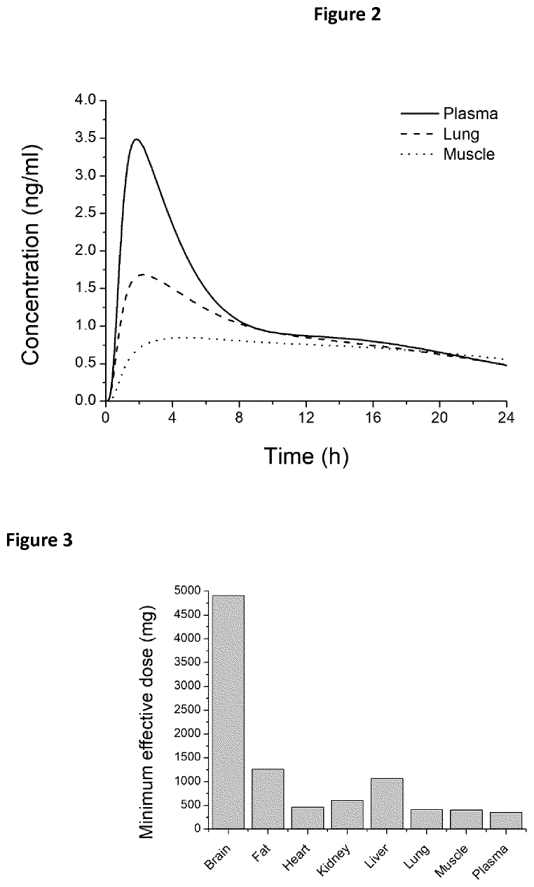

Simulated concentration profile of cordycepin in humans. PK-Sim was used to estimate the PK profile of cordycepins in the organs of a human subject. FIG. 2 shows the 24-hr concentration profile of cordycepin in plasma, lung, and muscle tissues (for illustration purpose only). The whole set of organ specific PK data expressed in AUCs are brain, fat, heart, kidney, liver, lung, muscle, and small and large intestinal lumen is shown in Table 3. Note that the AUC unit for intestinal lumen is mg*h/ml and ng*h/ml for the rest. A quick comparison shows our simulation (denoted by PK-Sim) yields tₘₐₓ=1.85 hours and t_{1/2}=7.93 hours similar to the clinical study reported by Kim et al. 2022. In addition, the simulation using a rat model produces plasma concentration profile like the in vivo study by Lee et al (Lee, Radhi et al. 2019) (data not shown). Therefore, even though we cannot completely validate the simulation result, this result seems reasonable.

Cordycepin seems stored in muscle. FIG. 2 shows an interesting feature. According to the simulation, cordycepin seems to be stored in muscle and released slowly. The half-life of cordycepin in muscle is 16.31 hours while it is 7.93 hours in plasma.

Estimated minimum effective dose of purified cordycepin required to inhibit SARS-CoV-2 virus. Based on the IC₅₀ of cordycepin against SARS-CoV-2 virus (Rabie 2022), Eq. (4) and AUC of all organs from Table 3, we calculated the minimum effective oral doses of cordycepin that can inhibit 50% of infectious virus in the corresponding organs. The results are shown in FIG. 3. FIG. 3 shows that it requires 4905.66 mg of cordycepin to inhibit 50% of viruses in the brain tissue, around 1200.4 mg for fat and liver and 355 mg for plasma, 463 mg for heart, 605 mg for kidney, 414 mg for lung, and 400 mg for muscle.

Therefore, we deduce that the recipe required consists of at least 4905.66 mg of cordycepin to achieve a system wide effect in human to deal with various complications from COVID-19. However, a dose between 400 to 600 mg of cordycepin will provide an adequate clinical response as effective concentrations are achieved in plasma, fat, heart, kidneys, liver, lung, muscles, and lumen of small and large intestine.

TABLE 3
Total body clearance, bioavailability, AUC₀₋₂₄¹ in plasma, and 7 other
organs and intestinal lumen (oral dose of cordycepin = 1 mg).

| | | | | | | | | | | AUC₀₋₂₄¹ (mg*h/ml) | |
|---|---|---|---|---|---|---|---|---|---|---|---|
| Total body | | | | | | | | | | Small | Large |
| clearance | Bioavailability | AUC₀₋₂₄¹ (ng*h/ml) | | | | | | | | intestinal | intestinal |
| (ml/min) | (%) | Plasma | Brain | Fat | Heart | Kidney | Liver | Lung | Muscle | lumen | lumen |
| 380.18 | 77.5 | 33.99 | 2.46 | 9.59 | 26.02 | 19.93 | 11.32 | 29.09 | 29.97 | 0.008 | 1.07 |

In summary, we have utilized in vitro and in silico data to set up a PK model and study ADME of cordycepin in humans. Subsequently, we used the pharmacokinetic (PK) model to estimate AUCs of cordycepin for each organ in humans. Additionally, we employed the in vitro IC₅₀ value for SARS-CoV-2 inhibition to predict and calculate the minimum effective dose of cordycepin required to inhibit 50% of the SARS-CoV-2 virus (Eq. (4)). Our calculations suggest that the minimum clinically effective dose of cordycepin is approximately 500 mg.

### Example 3

The objective of this example is to measure the chemical profile of an extract of Cordyceps militaris, which was evaluated in a preliminary study in humans.

Analysis of nucleosides and other bases in the extract 100 mg of each sample were dissolved in 0.3% formic acid at a concentration of 10.0 mg/ml with sonication for 1 hour at room temperature. The samples were then clarified by centrifugation, filtered, and directly injected onto HPLC. Detailed procedures are described in SCI-TMS 001. Samples were run in duplicates. The quantities of Uracil, Hypoxanthine, Uridine, Thymine, Adenine, Adenosine, and Cordycepin were determined using external standards (obtained from Sigma) by HPLC-DAD absorbance detection at 260 nm and identity was confirmed by retention time of each standard (FIG. 4 and Table 4).

TABLE 4
Nucleosides and bases in the extract.

| Sample Nr. | C (% by weight) |
|---|---|
| Uracil | 0.0114 ± 0.0006 |
| Hypoxanthine | 0.0485 ± 0.0029 |
| Uridine | 0.1540 ± 0.0136 |
| Thymine | 0.0088 ± 0.0008 |
| Adenine | 0.0255 ± 0.0017 |
| Adenosine | 1.3466 ± 0.0927 |
| Cordycepin | 3.0334 ± 0.1296 |

### Quantification of Polysaccharides

Extraction: 100 mg of each sample were dissolved in hot water (LC-MS grade) at a concentration of 10.0 mg/ml with sonication for 1 hour at 80° C. The samples were then clarified by centrifugation and filtered if needed.

Hydrolysis: Samples were hydrolyzed with concentrated HCl for 4 hours at 100-105° C. to get the corresponding monosaccharides.

Derivatization: All samples were treated with PMP, 1-phenyl-3-methyl-5-pyrazolone, as a derivatizing agent, for 4 hours at 68-70° C.

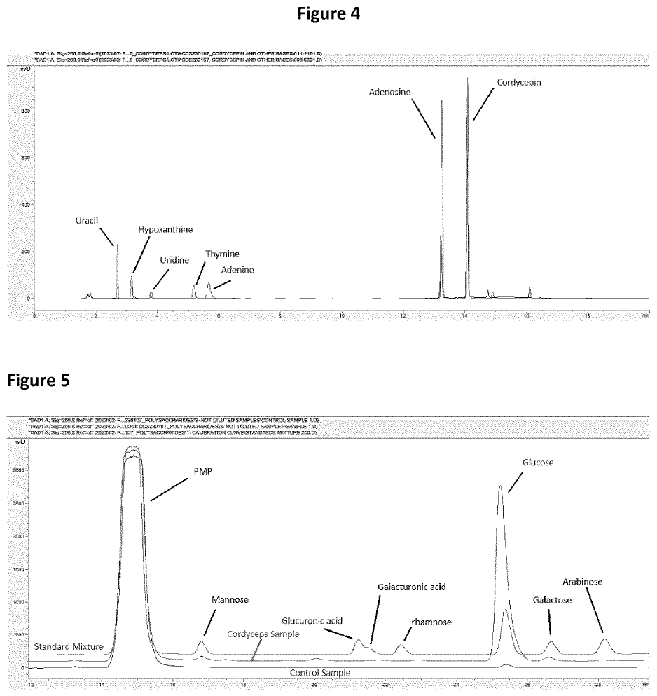

HPLC-DAD: All samples were filtered before injection and run in duplicates. The quantities of mannose, glucuronic acid, galacturonic acid, rhamnose, glucose, galactose, and arabinose were determined using external standards (obtained from Sigma) by HPLC-DAD absorbance detection at 250 nm and versus a control sample (normal sample but not hydrolyzed), and the identity was confirmed by retention time of each standard (after same treatment as in samples) (SCI-TMS-009-v2). Total polysaccharides were calculated as a sum of all monosaccharides and the value was corrected due to polysaccharides' hydrolysis (FIG. 5 and Table 5).

TABLE 5
Composition of polysaccharides in the extract

| Sample Nr. | C (% by weight) |
|---|---|
| Mannose | 0.6634 ± 0.1724 |
| Glucuronic acid | 0.3945 ± 0.0746 |
| Galacturonic acid | 0.3004 ± 0.1185 |
| Rhamnose | 0.3693 ± 0.1338 |
| Glucose | 39.7863 ± 8.7298 |
| Galactose | 0.5497 ± 0.1933 |
| Arabinose | 0.1534 ± 0.0280 |
| SUM (Total Polysaccharides) | 42.2170 |
| Corrected Value | 37.9953 |

Results from these studies clearly showed that adenosine, cordycepin and polysaccharides are the major components in the extract. Based on the computational analysis, it is reasonable to deduce that these three compounds are responsible for Cordyceps militaris' anti-COVID and anti-long COVID activity.

### Separation of Polysaccharides

Polysaccharides in Cordyceps militaris are separated using the polyacrylamide gel electrophoresis separation of polysaccharides (PS-PAGE) method. Briefly, Crude Cordyceps militaris Extract (CME) was prepared from the raw dried mushroom powder, using the procedure described by Dong, Hu et al. (2015) with slight modifications.

The Crude extract was used to prepare the polysaccharides' extract. This is accomplished by separating the proteins using SEVAG reagent and followed by polysaccharides precipitation with ethanol (Dong, Hu et al. 2015). The polysaccharides' residues were lyophilized and kept in freezer before analysis.

Polysaccharides were derivatized using 2-aminoacridone (AMAC), a fluorescent agent. The procedure described by Calabro, Benavides et al. (2000) was used with slight modifications.

PS-PAGE was carried out following the procedure described by A.L. Cunha et al. (2015) with slight modifications.

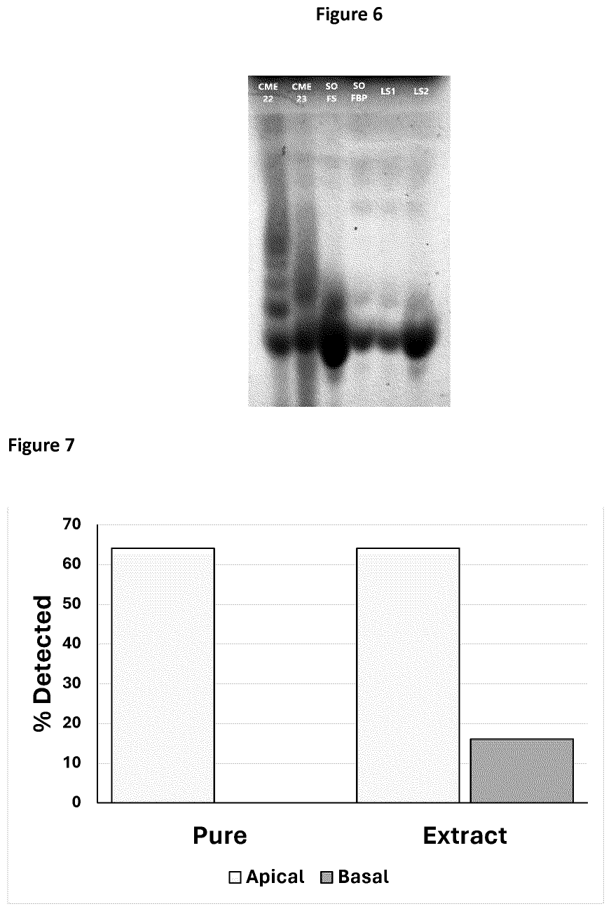

The polysaccharides of six Cordyceps militaris samples, collected from various sources, were separated using this method (FIG. 6). This method will be used as a quality control to ensure uniformity of the distribution of the polysaccharides.

### Example 4

The objective of this example is to evaluate potential increase in cordycepin oral bioavailability by the constituents of the extract.

The oral bioavailability of cordycepin is extremely low when given as a pure compound (Lee, Radhi et al. 2019). This is at least in part due to rapid elimination by deaminases in intestinal cells.

Methods: Transepithelial wells with a 1 μm pore size and 0.3 cm² surface area were preincubated with DMEM-F12 media with 10% FBS and 10 μg/ml penicillin/streptomycin at 37° C. in 5% CO₂ for one hour. CaCo2 cells in the same media were seeded at a density of 50,000 cells. The cells were maintained under these conditions throughout the experiment. Media was changed every 2 to 3 days, and TEER values were monitored from day 10 onward. At day 18, cells with TEER values exceeding 200 ohm/cm² were incubated in PAB buffer for 20 minutes: 1 ml in basolateral and 0.5 ml in apical side. Samples of 200 μg/ml cordycepin, and 200 μg/ml cordycepin in a standardized cordyceps extract were prepared in PAB buffer, which was exchanged for the apical buffer. Fifty microlitre samples of both apical and basolateral compartments were acquired initially and at 1 hour intervals. The samples were immediately added to 450 μl of methanol and quantified on an Agilent 4910 LC-mass spectrometer. Cordycepin was measured by direct injection in SMS mode and integrated values were compared against standard curves prepared in the same manner as test samples.

Results: As shown in FIG. 7, no detectable cordycepin was measured on the basal side suggesting the permeability of cordycepin is negligible when a solution of pure cordycepin was evaluated. This observation is consistent with that reported by Lee, Radhi et al. (2019). Rapid deamination during the permeation process could be one of the major reasons. The implication of this observation is a lack of meaningful bioavailability of cordycepin when it is administered orally in humans, suggesting cordycepin is unlikely to be effective in eradicating the virus on contact systemically.

The extract on the other hand showed significant permeability (Pₐₚₚ value of 3.76×10⁻⁶ cm/s), suggesting the oral bioavailability of cordycepin is enhanced by the constituents of the extract.

This set of results showed that, in addition to potential pharmacodynamic interactions among cordycepin and adenosine, there could be other constituents that could reduce the elimination and increase the bioavailability of cordycepin in humans. This may include adenosine and other adenosine analogs.

Systemic availability of cordycepin is important because cordycepin must gain access to the site of action to elicit its pharmacological activities, which include anti-viral activities in the lung.

### Example 5

The purpose of this example is to evaluate the effects of a Cordyceps militaris extract in human subjects who were tested either COVID positive (within 30 days) or suffering from the lingering symptoms of COVID three months after a positive diagnosis of COVID (WHO definition of Long COVID).

Specifically, the quantity of the three major components, adenosine, cordycepin and polysaccharides are 1.3, 3.0 and 38% (weight percent), respectively.

This extract (SCI-2213) was made into 300 mg capsules, each capsule contains 4.0 mg of adenosine, 9.0 mg of cordycepin and 114 mg of polysaccharides.

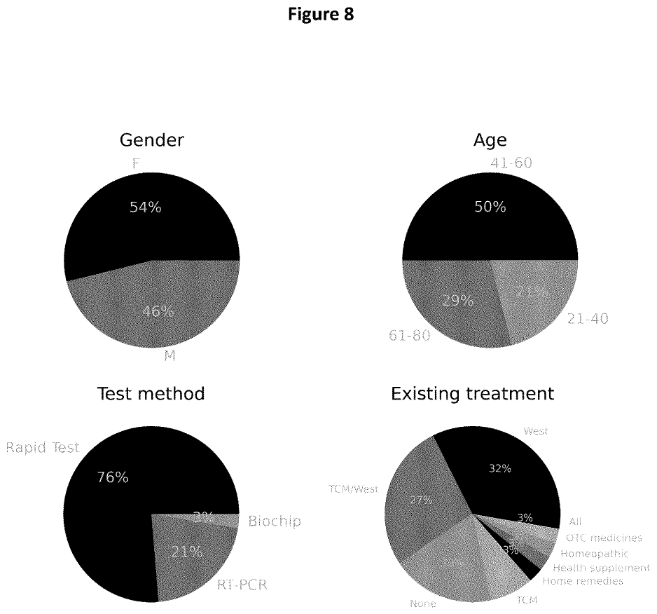

Forty subjects were recruited into a preliminary study, 18 male and 22 female, and aging ranging from 20 years to over 80 years of age (FIG. 8).

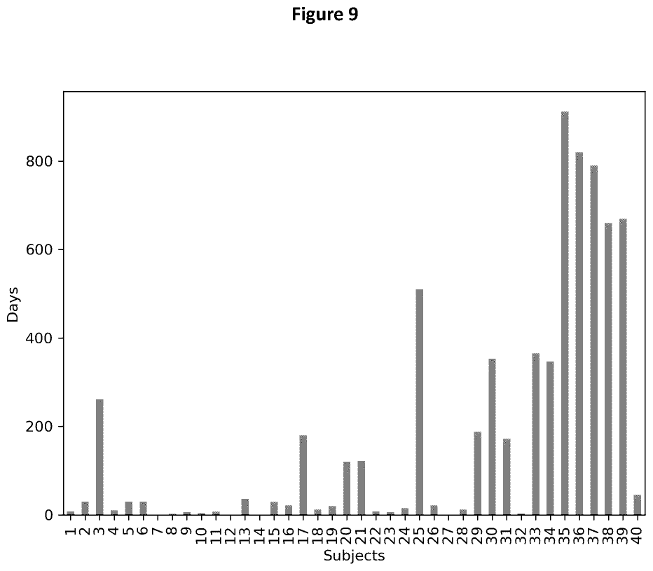

FIG. 9 shows the distribution of subjects in terms of time after a positive COVID test. These participants consist of acute COVID and long COVID sufferers.

The subjects were instructed to take two capsules, two times a day on an empty stomach for a period no shorter than seven days. Each subject was required to fill up a questionnaire before and daily during the study. The questionnaire involves the description of 11 COVID and long COVID symptoms. These symptoms were fever, cough, phlegm, headache, body ache, tiredness, brain fog, quality of sleep, hearing, taste, and smell. In a scale of 1 to 10, 10 is associated with no symptoms and 1 is the most severe discomfort experienced by the subject.

### Data Analysis

Data were preprocessed before the final analysis. For each symptom, if the score is 5 or higher, the data will be excluded from analysis because the margin of improvement would not be high enough for statistical analysis.

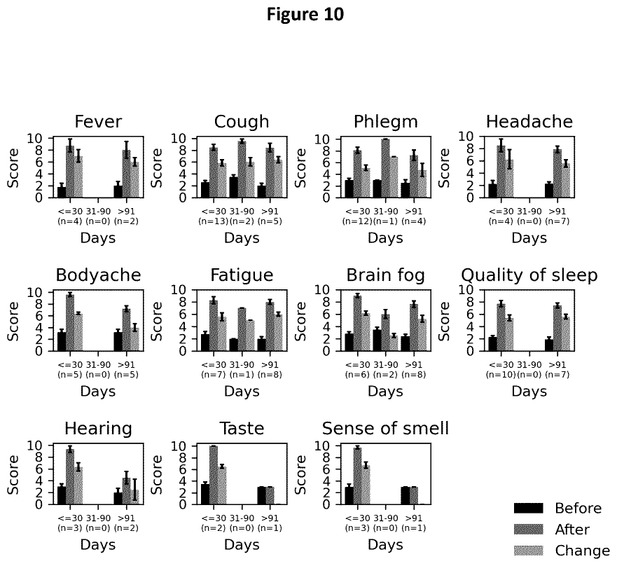

The mean score for each symptom before the subject started taking the extract ranged from 3 to 5. The mean response for all symptoms after treatment were all above 8 (FIG. 10). The data indicated that SCI-2213 has significant effects on alleviating symptoms associated with COVID and long COVID. It appears that the duration after positive diagnosis of COVID has little effect on the efficacy of SCI-2213.

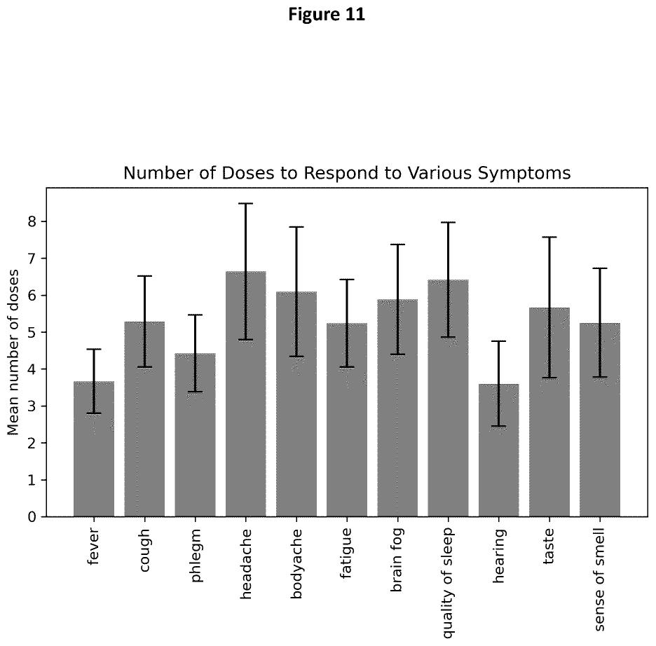

The mean dose to achieve this significant response is 5.29, suggesting that the participants felt significant relief of their symptoms in less than 2 days on average (FIG. 11).

A male subject who started consuming SCI-2213 one day after a positive diagnosis of COVID. He did not respond to the treatment for two days. Instead, his condition worsened. Specifically, his fever was higher, his extremities became painful, and his cough worsened. After his dose was elevated to four capsules two times a day, his condition was significantly improved within 24 hours. This observation suggests a dose related response to SCI-2213.

In the absence of a diagnosis for long COVID, let alone a treatment, SCI-2213 could be a solution for providing relief for those who suffer mild to moderate COVID and long COVID symptoms. SCI-2213 will fulfill the unmet need the world is waiting for.

### CONCLUSIONS

In this disclosure, a combination therapy was developed to treat COVID and long COVID using the processes developed by Tam, Tseng et al. (2022). Initial observations in patients are encouraging as the response rate to major symptoms is as high as 90%. In one embodiment, the combination comprises SCI-2213 with a synergistic combination of its ingredients, each in preferred dosages.

### REFERENCES

The following references are indicative of the level of skill of one skilled in the art, and are incorporated herein by reference in their entirety, where permitted.
Amstutz, A., B. Speich, F. Mentre, C. S. Rueegg, D. Belhadi, L. Assoumou, C. Burdet, S. Murthy, L. E. Dodd, Y. Wang, K. A. O. Tikkinen, F. Ader, M. Hites, M. Bouscambert, M. A. Trabaud, M. Fralick, T. C. Lee, R. Pinto, A. Barratt-Due, F. Lund-Johansen, F. Muller, O. P. O. Nevalainen, B. Cao, T. Bonnett, A. Griessbach, A. Taji Heravi, C. Schonenberger, P. Janiaud, L. Werlen, S. Aghlmandi, S. Schandelmaier, Y. Yazdanpanah, D. Costagliola, I. C. Olsen and M. Briel (2023). “Effects of remdesivir in patients hospitalised with COVID-19: a systematic review and individual patient data meta-analysis of randomised controlled trials.” Lancet Respir Med.
Badulescu, O. V., P. D. Sirbu, N. Filip, G. Bordeianu, E. Cojocaru, C. C. Budacu, M. C. Badescu, I. Bararu-Bojan, B. Veliceasa and M. Ciocoiu (2022). “Hereditary Thrombophilia in the Era of COVID-19.” Healthcare (Basel) 10(6).
Karlowitz, R., M. L. Stanifer, J. Roedig, G. Andrieux, D. Bojkova, M. Bechtel, S. Smith, L. Kowald, R. Schubert, M. Boerries, J. Cinatl, Jr., S. Boulant and S. J. L. van Wijk (2022). “USP22 controls type 11 interferon signaling and SARS-CoV-2 infection through activation of STING.” Cell Death Dis 13(8): 684.
Kaymakci, M. A. and E. M. Guler (2020). “Promising Potential Pharmaceuticals from the Genus Cordyceps for COVID-19 Treatment: A Review Study.” Bezmialem Science 8(3): 140-144.
Kuo, C. F., C. C. Chen, C. F. Lin, M. S. Jan, R. Y. Huang, Y. H. Luo, W. J. Chuang, C. C. Sheu and Y. S. Lin (2007). “Abrogation of streptococcal pyrogenic exotoxin B-mediated suppression of phagocytosis in U937 cells by Cordyceps sinensis mycelium via production of cytokines.” Food Chem Toxicol 45(2): 278-285.
Lee, J. B., M. Radhi, E. Cipolla, R. D. Gandhi, S. Sarmad, A. Zgair, T. H. Kim, W. Feng, C. Qin, C. Adrower, C. A. Ortori, D. A. Barrett, L. Kagan, P. M. Fischer, C. H. de Moor and P. Gershkovich (2019). “A novel nucleoside rescue metabolic pathway may be responsible for therapeutic effect of orally administered cordycepin.” Sci Rep 9(1): 15760.
Lee, J. S., D. S. Kwon, K. R. Lee, J. M. Park, S. J. Ha and E. K. Hong (2015). “Mechanism of macrophage activation induced by polysaccharide from Cordyceps militaris culture broth.” Carbohydr Polym 120: 29-37.
Liu, J. Y., C. P. Feng, X. Li, M. C. Chang, J. L. Meng and L. J. Xu (2016). “Immunomodulatory and antioxidative activity of Cordyceps militaris polysaccharides in mice.” Int J Biol Macromol 86: 594-598.
Lu, R. J., P. Shirvani and M. F. Holick (2022). “A Novel Immunomodulatory Mechanism by Which Vitamin D Influences Folate Receptor 3 Expression to Reduce COVID-19 Severity.” Anticancer Res 42(10): 5043-5048.
Mao, Y. H., F. L. Song, Y. X. Xu, A. X. Song, Z. M. Wang, M. Z. Zhao, F. He, Z. Z. Tian and Y. Yang (2022). “Extraction, Characterization, and Platelet Inhibitory Effects of Two Polysaccharides from the Cs-4 Fungus.” Int J Mol Sci 23(20).
Miao, M., W. Q. Yu, Y. Li, Y. L. Sun and S. D. Guo (2022). “Structural Elucidation and Activities of Cordyceps militaris-Derived Polysaccharides: A Review.” Front Nutr 9: 898674.
Namkoong, H., et al. (2022). “DOCK2 is involved in the host genetics and biology of severe COVID-19.” Nature 609(7928): 754-760.
Ohta Y., L. J.-B., Hayashi K., Fujita A., Park D. K., Hayashi T., (2007). “In Vivo Anti-influenza Virus Activity of an Immunomodulatory Acidic Polysaccharide Isolated from Cordyceps militaris Grown on Germinated Soybeans.” J. Agric. Food Chem. 55: 6.
Poulin, P. and F. P. Theil (2002). “Prediction of pharmacokinetics prior to in vivo studies. 1. Mechanism-based prediction of volume of distribution.” J Pharm Sci 91(1): 129-156.
Rabie, A. M. (2022). “Potent Inhibitory Activities of the Adenosine Analogue Cordycepin on SARS-CoV-2 Replication.” ACS Omega 7(3): 2960-2969.
Redin, C., C. W. Thorball and J. Fellay (2022). “Host genomics of SARS-CoV-2 infection.” Eur J Hum Genet 30(8): 908-914.
Singh, M., R. Tulsawani, P. Koganti, A. Chauhan, M. Manickam and K. Misra (2013). “Cordyceps sinensis increases hypoxia tolerance by inducing heme oxygenase-1 and metallothionein via Nrf2 activation in human lung epithelial cells.” Biomed Res Int 2013: 569206.
Tam, Y. K., C.-Y. Tseng and T. Bjorndahl (2022). A Pharmaceutical Platform Technology For Drug Discovery And Consumer Health Product Development. WIPO/PCT WO 2023/021472 A1.
Verma, A. K. (2020). “Cordycepin: a bioactive metabolite of Cordyceps militaris and polyadenylation inhibitor with therapeutic potential against COVID-19.” J Biomol Struct Dyn: 1-8.
Wang, W., L. Xu, J. Su, M. P. Peppelenbosch and Q. Pan (2017). “Transcriptional Regulation of Antiviral Interferon-Stimulated Genes.” Trends Microbiol 25(7): 573-584.
Yang, F. Q., D. Q. Li, K. Feng, D. J. Hu and S. P. Li (2010). “Determination of nucleotides, nucleosides and their transformation products in Cordyceps by ion-pairing reversed-phase liquid chromatography-mass spectrometry.” J Chromatogr A 1217(34): 5501-5510.
Zivan, T., R. L. Ruiz, A. Martinez and B. B. Pavri (2023). “Safety of Adenosine for the Treatment of Supraventricular Tachycardia in Hospitalized Patients with COVID-19 Pneumonia.” J Clin Med 12(3).
Calabro, A., M. Benavides, M. Tammi, V. C. Hascall and R. J. Midura (2000). “Microanalysis of enzyme digests of hyaluronan and chondroitin/dermatansulfate by fluorophore-assisted carbohydrate electrophoresis (FACE).” Glycobiology 10(3): 273-281.
Dong, Y., S. Hu, C. Liu, Q. Meng, J. Song, J. Lu, Y. Cheng, C. Gao, Y. Liu, D. Wang and L. Teng (2015). “Purification of polysaccharides from Cordyceps militaris and their antihypoxic effect.” Mol Med Rep 11(2): 1312-1317.
Dubhashi, S., S. Sinha, S. Dwivedi, J. Ghanekar, S. Kadam, P. Samant, V. Datta, S. Singh, I. H. Chaudry, P. Gurmet, H. Kelkar, R. Mishra, S. Galwankar and A. Agrawal (2023). “Early Trends to Show the Efficacy of Cordyceps militaris in Mild to Moderate COVID Inflammation.” Cureus.
Jedrejko, K. J., J. Lazur and B. Muszynska (2021). “Cordyceps militaris: An Overview of Its Chemical Constituents in Relation to Biological Activity.” Foods 10(11).
Lee, J. B., M. Radhi, E. Cipolla, R. D. Gandhi, S. Sarmad, A. Zgair, T. H. Kim, W. Feng, C. Qin, C. Adrower, C. A. Ortori, D. A. Barrett, L. Kagan, P. M. Fischer, C. H. de Moor and P. Gershkovich (2019). “A novel nucleoside rescue metabolic pathway may be responsible for therapeutic effect of orally administered cordycepin.” Sci Rep 9(1): 15760.
Mao, Y. H., F. L. Song, Y. X. Xu, A. X. Song, Z. M. Wang, M. Z. Zhao, F. He, Z. Z. Tian and Y. Yang (2022). “Extraction, Characterization, and Platelet Inhibitory Effects of Two Polysaccharides from the Cs-4 Fungus.” Int J Mol Sci 23(20).
Rabie, A. M. (2022). “Potent Inhibitory Activities of the Adenosine Analogue Cordycepin on SARS-CoV-2 Replication.” ACS Omega 7(3): 2960-2969.

1. A composition for the treatment of COVID and/or long COVID, comprising cordycepin, adenosine and a Cordyceps fungi extract comprising polysaccharides.

2. The composition of claim 1 wherein the ratio of the polysaccharide component to cordycepin is between about 3:1 to about 40:1.

3. The composition of claim 1, wherein the ratio of cordycepin to adenosine is between about 1:5 to about 10:1.

4. The composition of claim 1, wherein either or both of cordycepin and adenosine is extracted from Cordyceps.

5. The composition of claim 1, wherein either or both of cordycepin and adenosine is produced synthetically.

6. The composition of claim 1 which is in its pure form or in the form of a standardized extract.

7. An oral dosage form comprising the composition of claim 1, optionally comprising a pharmaceutically acceptable carrier.

8. The oral dosage form of claim 7 which is a capsule, tablet, solution, suspension, powder, or a nano-encapsulated dosage form.

9. A method of treating COVID or Long COVID by administering a therapeutically effective amount of the composition of claim 1, or an oral dosage form of claim 7.

10. The method of claim 9 wherein the polysaccharide and cordycepin is administered in a ratio of between about 3:1 to about 40:1.

11. The method of claim 10 wherein the ratio is about 20:1 to about 35:1.

12. The method of claim 9 wherein cordycepin and adenosine are administered in a ratio of between about 1:5 to about 10:1.

13. The method of claim 12, wherein the ratio is about 2:1 to about 5:1.

14. The method of claim 9, wherein a daily dose comprises a daily dose of polysaccharides of between about 200 to about 800 mg, a daily dose of cordycepin between about 15 to about 50 mg, and/or a daily dose of adenosine is between about 4 to about 50 mg, for a human having a body mass of 70 kg, or an equivalent daily dose calculated per kg of body mass of a human.

15. The method of claim 14 wherein the daily dose comprises one or more doses of a 200-400 mg oral dosage form, comprising about 1.0 to about 15.0 mg of adenosine, about 5.0 mg to about 15.0 mg of cordycepin, and/or about 50 mg to about 200 mg of polysaccharide.
